(19) United States
(12) Patent Application Publication
(10) Pub. No.: US 2011/0177572 A1
(43) Pub. Date: Jul. 21, 2011

(54) Method of Cultivating Fermentative Yeast Used for Xylose Fermentation of Non-Detoxified Lignocellulosic Hydrolysate

(75) Inventors: Jia-Baau Wang, Longtan Shiang (TW); Lee-Chung Men, Longtan Shiang (TW); Wen-Song Hwang, Longtan Shiang (TW); Wen-Hua Chen, Longtan Shiang (TW); Sheng-Shieh Chen, Longtan Shiang (TW); Wen-Heng Chen, Longtan Shiang (TW); Ting-Hsiang Lin, Longtan Shiang (TW); Chiung-Fang Huang, Longtan Shiang (TW); Sheng-Hsin Chou, Longtan Shiang (TW); Teng-Chieh Hsu, Longtan Shiang (TW); Gia-Luen Guo, Longtan Shiang (TW)

(73) Assignee: ATOMIC ENERGY COUNCIL-INSTITUTE OF NUCLEAR ENERGY RESEARCH, Taoyuan County (TW)

(21) Appl. No.: 12/689,809

(22) Filed: Jan. 19, 2010

(51) Int. Cl. C12P 7/06; C12N 1/16

(52) U.S. Cl. 435/161; 435/255.1

(57) ABSTRACT

A hydrolysate-adapted yeast, Pichia stipitis INER 1128, is cultivated according to the present invention. The adapted yeast can effectively convert xylose into ethanol in lignocellulosic hydrolysate, which is not even detoxified. Well ethanol yield is obtained while xylose is not wasted and thus cost is reduced.

### FIELD OF THE INVENTION

The present invention relates to cultivating a fermentative yeast; more particularly, relates to cultivating a fermentative yeast, Pichia stipitis INER 1128, through an adaptation process to be used for xylose fermentation of a non-detoxified hydrolysate or hydrolysate with overliming treatment for converting xylose into ethanol with a yield over 90%.

### DESCRIPTION OF THE RELATED ARTS

Bioethanol is a potential fuel for replacing gasoline. Now commercial bioethanol is mainly obtained from grain and sugar cane, where starch and sucrose can be converted into ethanol through fermentation after a simple preparation. A wine making yeast like Saccharomyces cerevisiae is usually used for fermentation and thus obtained a well ethanol yield.

However, using grain and sugar cane for ethanol production is always arguable, since it may affect provisions for human. Hence, lignocellulosic materials like wood, bagasse, rice straw, corn stover, wheat straw, silvergrass and paper wastes are considered the most potential in future for ethanol production due to the amounts in abundance, the diversity of source and non-conflictive for food supply.

In general, the lignocellulosic material has 60% to 80% of cellulose and hemicellulose along with 15% to 25% of lignin. After cellulose and hemicellulose are converted into hexose (mainly from glucose) and pentose (mainly from xylose) through a saccharification process, these sugars can be further converted into ethanol through fermentation. Furthermore, for the progress of lignocellulosic materials-to-ethanol, a thermo-chemical pretreatment such as dilute-acid hydrolysis or acid-catalyzed steam explosion is usually used to convert hemicellulose into xylose. These thermo-chemical pretreatments, operated under a high temperature and a high pressure, often contained lignocellulosic materials and water solution with 1% to 2% (w/w) of a dilute sulfuric acid. The liquid obtained after the pretreatment is so-called xylose-rich hydrolysate or hydrolysate, which has more of sulfate owing to the dilute sulfuric acid added; This feature may reduce the ability of converting xylose into ethanol with the fermentative yeast. In addition, using dilute acid pretreatment with different operation conditions would often produce various amounts of fermentative inhibitors like acetic acid, furfural and hydroxymethyl furfura.

Hence, the xylose-rich hydrolysate obtained is usually processed through an overliming treatment to be detoxified by means of removing furfural and sulfate. However, the overliming operation may lead xylose lost and simultaneously result in the production of gypsum, which would require extra equipments and expense for handling in the progress of converting xylose into ethanol.

In addition, the wine making yeast used is unable to convert pentose into ethanol. Only few natively yeast, like Pichia stipitis and Candida shehatae, have shown well abilities on converting xylose into ethanol. To date, lignocellulosic materials used for study on converting xylose to into ethanol by Pichia stipitis etc., include corn stover, corn cob, hardwood, softwood, water hyacinth, wheat straw, sunflower seed hull and rice straw. However, in addition to a research “Production of ethanol from corn stover hemicellulose hydrolyzate using Pichia stipitis”, by Agbogbo, F. K. and K. S. Wenger, Journal of Industrial Microbiology and Biotechnology 34, 723-727, 2007, which obtained an ethanol yield of 85% from fermentation of corn stover hydrolysate by Pichia stipitis. Ethanol yields are all below 75% for others.

Besides, the hydrolysate almost has to be detoxified through overliming process for obtaining a maximal ethanol yield as mentioned above. Only in “Fermentation of acid-pretreated corn stover to ethanol without detoxification using Pichia stipitis” by Agbogbo al., Appl. Biochem. Biotechnol, 2007, Pichia stipitis is used to convert xylose into ethanol in a non-detoxified corn stover hydrolysate, but the ethanol yield just reached 83%. Hence, it is hard to reduce xylose loss as well as to enhance the ethanol yield using prior arts, which are unable to fulfill all users' requests on actual use.

### SUMMARY OF THE INVENTION

The main purpose of the present invention is to cultivate fermentative yeast, Pichia stipitis INER 1128, through an adaptation process. This hydrolysate-adapted yeast is useful for converting xylose into ethanol in non-detoxified hydrolysate or hydrolysate with overliming treatment.

The second purpose of the present invention is to convert xylose into ethanol in the non-detoxified hydrolysate or hydrolysate with overliming treatment with an ethanol yield over 90%.

The third purpose of the present invention is to provide adapted yeast for converting xylose into ethanol with a high ethanol yield while xylose is not wasted and thus cost for converting xylose into ethanol is reduced.

To achieve the above purposes, the present invention is a method of cultivating a hydrolysate-adapted yeast used for fermentation of non-detoxified hydrolysate; it comprised the steps of: (a) mixing a non-detoxified hydrolysate and an synthetic medium with a volumetric ratio of 20:80 to obtain a mixed medium for culture, where the synthetic medium contains yeast extract and peptone; (b) adding xylose and glucose into the mixed medium to obtain a xylose concentration of 30 grams per liter (g/L) and a glucose concentration of 10 g/L, where a fermentative yeast for converting xylose into ethanol is cultivated in the mixed medium; (c) after sub-culturing the yeast for two generations, gradually increasing the volumetric ratio of the hydrolysate to the synthetic medium in turn at 35:65, 50:50, 60:40 and 70:30 with maintaining a final xylose concentration and glucose concentration as mentioned above; and (d) finally sub-culturing the hydrolysate-adapted yeast in the non-detoxified hydrolysate for at least 60 generations. Accordingly, fermentative yeast after adaptation used for xylose fermentation of non-detoxified hydrolysate is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow chart showing the preferred embodiment according to the present invention;

FIG. 2 is the flow chart showing the dilute acid pretreatment;

FIG. 3 is the flow chart showing the overliming process of the hydrolysate;

FIG. 4 is the flow chart showing the neutralization operation of the hydrolysate;

FIG. 5 is the illustration showing the ethanol yields from fermentation of hydrolysate with over-liming treatment in flask;

FIG. 6 is the illustration showing the xylose concentration trends from fermentation of the hydrolysate with overliming treatment in flask;

FIG. 7 is the illustration showing the ethanol yield and xylose concentration trend from fermentation of hydrolysate with overliming treatment in a 5 L fermentor; and

FIG. 8 is the illustration comparing the ethanol yields from fermentation of the non-detoxified hydrolysate and hydrolysate with overliming treatment.

### DESCRIPTION OF THE PREFERRED EMBODIMENT

The following description of the preferred embodiment is provided to understand the features and the structures of the present invention.

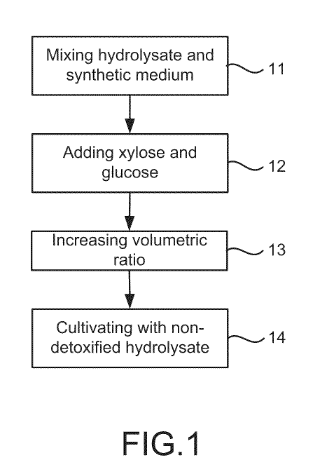

Please refer to FIG. 1, which is a flow chart showing a preferred embodiment according to the present invention. As shown in the figure, the present invention cultivates yeast for converting xylose into ethanol through adaptation used for fermentation of a non-detoxified lignocellulosic hydrolysate, comprising the following steps:

(a1) Mixing xylose-rich hydrolysate and synthetic medium 11: A xylose-rich hydrolysate and a synthetic medium is mixed under a volumetric ratio of 20:80 to obtain a mixed medium for culture, where the xylose-rich hydrolysate is not detoxified and the synthetic medium contained yeast extract and peptone.

(b1) Adding xylose and glucose 12: Xylose and glucose are separately added into the mixed medium to obtain a final xylose concentration of 30 grams per liter (g/L) and a glucose concentration of 10 g/L. Fermentative yeast, Pichia stipitis (ATCC 58785; BCRC21777), is cultivated in the mixed medium.

(c1) Increasing volumetric ratio 13: After the yeast is sub-cultured for two generations, the volumetric ratio of the xylose-rich hydrolysate to the synthetic medium is gradually increased from 20:80 to 70:30 along through 35:65, 50:50 and 60:40. Meanwhile, the final xylose concentration and the glucose concentration in mixed medium are maintained at a designed value as mentioned above. Following these steps, the fermentative yeast showed a high tolerance to the non-detoxified hydrolysate.

(d1) Cultivating with non-detoxified hydrolysate 14: The fermentative yeast is directly sub-cultured in the non-detoxified hydrolysate for at least 60 generations. A hydrolysate-adapted yeast, called Pichia stipitis INER 1128, is finally obtained, which grows well in a non-detoxified hydrolysate and has a well ethanol yield for xylose conversion.

Thus, with the above steps, a novel method of cultivating a hydrolysate-adapted yeast used for xylose fermentation of non-detoxified hydrolysate is obtained.

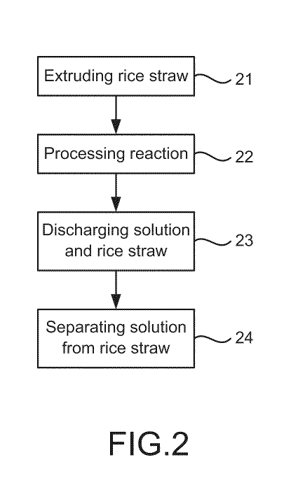

Please refer to FIG. 2, which is a flow chart showing a dilute acid treatment. As shown in the figure, a xylose-rich hydrolysate according to the present invention is processed through a dilute acid treatment, comprising the following steps:

(a2) Extruding rice straw 21: Rice straw is cut at a certain size to be extruded with specific volume of water in a twin screw extruder under a sulfuric acid of 3% (w/w), a screw rotated speed of 40 rotations per minute (rpm), a reaction temperature of 145 Celsius degrees (° C.), a reaction time of 20 minutes (min), and a mixing ratio of 50:100 for a dry weight of rice straw to a weight of the water.

(b2) Processing reaction 22: After destroying the structure of the rice straw by twin screw extruder, the rice straw with the liquid is thus put into an acid-catalyzed reactor with a steam inlet to process a reaction under a temperature of 130° C. while the mixing ratio of the dry weight of the rice straw to the weight of the liquid is reduced to 30:100 after steam introduced.

(c2) Discharging liquid and rice straw 23: After the liquid is boiled for 15 min with the rice straw under reaction temperature as mentioned above, the liquid is discharged along with the rice straw.

(d2) Separating liquid from rice straw 24: After the reaction, the liquid is separated from the rice straw with solid and liquid separation equipments. The liquid obtained is so-called xylose-rich hydrolysate or hydrolysate, having glucose, xylose, arabinose, acetic acid, furfural and hydroxymethyl furfural, mainly contained 20 g/L of xylose with a pH value between 1.0 and 1.6.

Thus, hydrolysate-adapted yeast, Pichia stipitis INER 1128, obtained according to the present invention is applicable to fermentation of a non-detoxified hydrolysate obtained through the dilute acid treatment, where the hydrolysate can be obtained from a lignocellulosic material of rice straw, bagasse, silvergrass, napiergrass, switchgrass, corn stover, wheat straw, wood, bamboo, water hyacinth or algae.

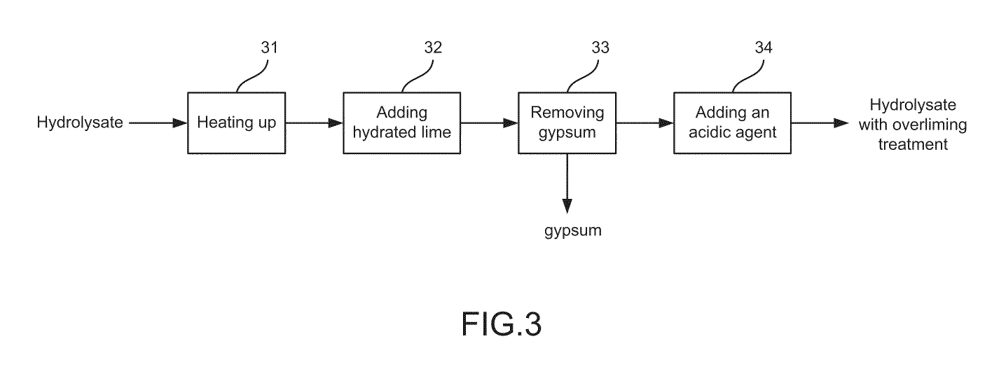

Please refer to FIG. 3, which is a flow chart showing overliming process of a xylose-rich hydrolysate. As shown in the figure, hydrolysate-adapted yeast obtained according to the present invention is applicable to fermentation of hydrolysate with overliming treatment, which is processed through the following steps:

(a3) Heating up 31: A xylose-rich hydrolysate is heated up to a temperature between 50° C. and 60° C.

(b3) Adding hydrated lime (calcium hydroxide) 32: The xylose-rich hydrolysate is added with hydrated lime to achieve a pH value between 9.0 and 11.0. Thus, the xylose-rich hydrolysate is detoxified with gypsum produced.

(c3) Removing gypsum 33: Then gypsum is removed from the xylose-rich hydrolysate with solid and liquid separation equipments. The xylose-rich hydrolysate obtained is free from furfural and sulfate.

(d3) Adding an acidic agent 34: An acidic agent is added to make the xylose-rich hydrolysate to be a weak acidity solution.

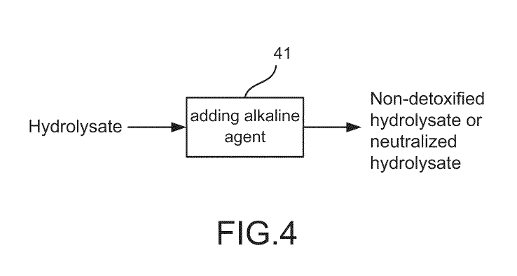

Please refer to FIG. 4, which is a flow chart showing neutralization process of a xylose-rich hydrolysate. As shown in the figure, a xylose-rich hydrolysate is directly added with an alkaline agent 41 to become a weak acidity solution having a pH value between 5.0 and 7.0 without detoxification. In the present invention, an overliming process for hydrolysate, including heating up, adding hydrated lime, separating solid and liquid, and adding acidic agent, is not required for neutralization process. Instead, the xylose-rich hydrolysate is only required to add an alkaline agent and thus make the xylose-rich hydrolysate to be a weak-acidity of solution

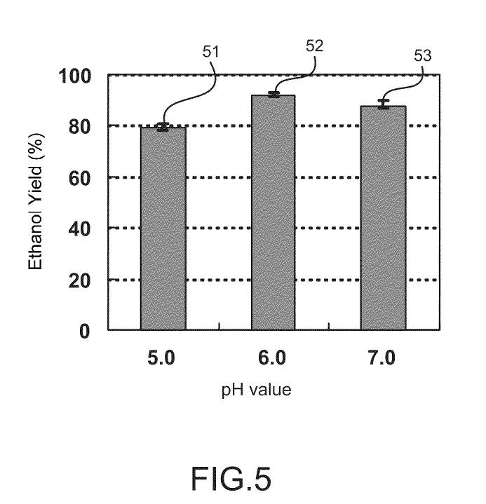

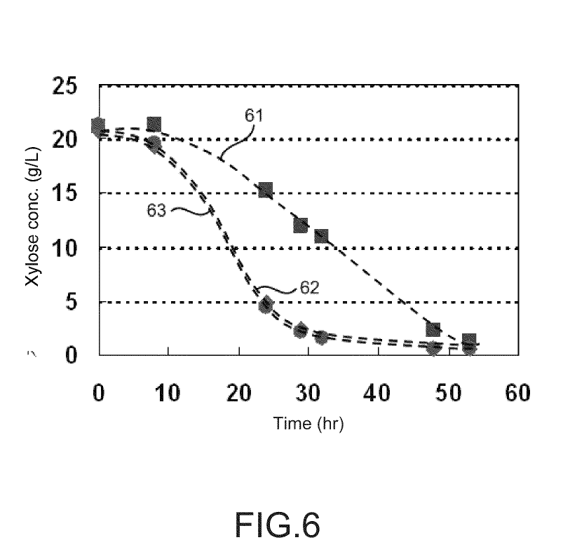

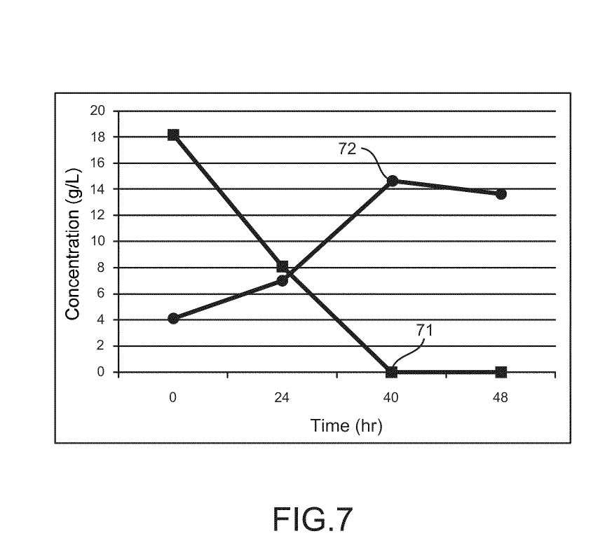

Please refer to FIG. 5 to FIG. 7, which are illustrations showing ethanol yields (FIG. 5) and the trends of xylose concentration (FIG. 6) from the fermentation of a hydrolysate with overliming treatment in flask; and an illustration showing an ethanol yield and xylose concentration trend from fermentation of the hydrolysate with overliming treatment in a 5 L fermentor (FIG. 7). As shown in the figures, a hydrolysate-adapted, Pichia stipitis INER 1128, obtained according to the present invention is applied to a rice straw hydrolysate with overliming and non-detoxified hydrolysate of rice straw. A shake flask is used for xylose fermentation with 50 milliliters (mL) of the xylose-rich hydrolysate under pH values of 5.0, 6.0 and 7.0, respectively. Ethanol yields are thus obtained under a temperature of 30° C., a shaking speed of 100 rpm in an incubator, and an inoculating amount of hydrolysate-adapted yeast between 0.4 g/L and 0.5 g/L.

As shown in FIG. 5, when the pH value is controlled at 5.0, the ethanol yield 51 is about 80%. When the pH value is controlled at 6.0 or 7.0, the ethanol yield 52,53 is improved to around 90%. It shows that, better ethanol yield of the hydrolysate-adapted yeast is obtained with increase of the pH value kept. That means the hydrolysate-adapted yeast obtained according to the present invention exhibits a well ethanol yield from the fermentation of xylose-rich hydrolysate.

As shown in FIG. 6, when the pH values are controlled at 5.0, 6.0 and 7.0 separately, the trend of xylose concentration is different. A xylose concentration trend 61 for the pH value controlled at 5.0 shows that xylose concentration is negligible after 54 hours (hr) of fermentation. The xylose concentration trends 62,63 for the pH values controlled at 6.0 and 7.0 show that xylose concentrations are negligible at 40 hr of fermentation. Therein, as shown in FIG. 7, when the pH value is controlled at 6.0 and an air inflow is 0.02 volumes per volume per minute (vvm) for a 5 L fermentor, xylose concentration 71 is negligible after 40 hr of fermentation while a highest ethanol yield 72 from xylose conversion at 92% is obtained. Conclusively, the hydrolysate-adapted yeast obtained according to the present invention shows a better ethanol yield and faster ethanol productivity with a shorter reaction time when being operated at a higher pH value.

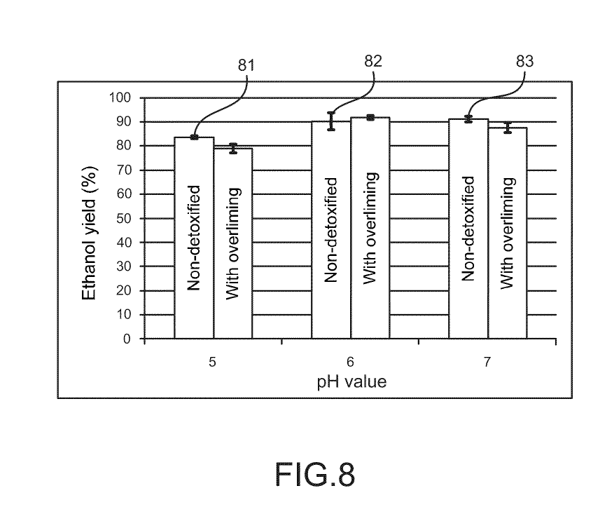

Please refer to FIG. 8, which is a illustration showing ethanol yields from fermentation of a non-detoxified hydrolysate and the hydrolysate with overliming treatment. As shown in the figure, non-detoxified hydrolysates are directly added with an alkaline agent to obtain pH values of 5.0, 6.0 and 7.0 separately. Then, a hydrolysate-adapted yeast, Pichia stipitis INER 1128, obtained according to the present invention is used for the fermentation of non-detoxified hydrolysates and the hydrolysate with overliming treatment. Ethanol yields for the non-detoxified hydrolysates are shown in the figure, where ethanol yields obtained are quite similar to that for hydrolysates with overliming treatment. When the pH value is 5.0, the corresponding ethanol yield is 80%. When the pH value is increased to 6.0 or 7.0, the maximal ethanol yield is achieved to 90%. It shows that, even though the hydrolysate is not detoxified through overliming process, a well ethanol yield is still obtained for the hydrolysate by using the adapted yeast obtained according to the present invention. That means the hydrolysate-adapted yeast obtained according to the present invention has developed tolerance to the fermentative inhibitors in hydrolysate. Furthermore, a procedure for xylose-rich hydrolysate fermentation is simplified and thus using the hydrolysate-adapted yeast obtained according to the present invention reduces the facility cost. Thus, the present invention is a method of cultivating a hydrolysate-adapted yeast used for fermentation of a non-detoxified hydrolysate, where a hydrolysate-adapted, Pichia stipitis INER 1128, is cultivated through adaptation to be used for fermentation of a non-detoxified hydrolysate with an ethanol yield of 90%.

Overall, the present invention is a method of cultivating a fermentative yeast used for fermentation a non-detoxified hydrolysate, where a hydrolysate-adapted yeast, Pichia stipitis INER 1128, is cultivated through adaptation to be used for fermentation of a non-detoxified hydrolysate or hydrolysate with overliming treatment for effectively converting xylose into ethanol with a yield over 90%. Moreover, xylose is not wasted and thus cost is reduced.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of cultivating fermentative yeast used for xylose fermentation of a non-detoxified hydrolysate, comprising steps of:
(a1) mixing a hydrolysate and a synthetic medium with an initially volumetric ratio to obtain a mixed medium for culture, said hydrolysate being non-detoxified, said synthetic medium having yeast extract and peptone;
(b1) obtaining a final xylose concentration and glucose concentration by separately adding xylose and glucose to cultivate a fermentative yeast in said mixed medium for culture;
(c1) after sub-culturing said fermentative yeast two generations, gradually increasing said volumetric ratio of said hydrolysate to said synthetic medium until a final volumetric ratio with maintaining said a final xylose concentration and said glucose concentration; and
(d1) directly sub-culturing said fermentative yeast a plurality of generations with said hydrolysate to obtain an hydrolysate-adapted yeast.

2. The method according to claim 1, wherein, in step (a1), said initially volumetric ratio is 20:80.

3. The method according to claim 1, wherein, in step (a1), said hydrolysate is obtained through a dilute acid pretreatment.

4. The method according to claim 3, wherein said dilute acid pretreatment comprises steps of:
(a2) cutting rice straw at a certain size to be extruded with a solution in a twin screw extruder under a certain condition;
(b2) after destroying a structure of said rice straw, putting said rice straw and said solution into an acid-catalyzed reactor with a steam inlet to process a reaction under a temperature while a mixing ratio of a dry weight of said rice straw to a weight of said solution is reduced;
(c2) after boiling said solution with said rice straw to react for a period of time, discharging said solution along with said rice straw; and
(d2) separating said solution from said rice straw with solid and liquid separation equipments.

5. The method according to claim 4, wherein, in step (a2), said certain condition includes a acid of 3% (w/w), a screw rotated speed of 40 rotations per minute (rpm), a reaction temperature of 145 Celsius degrees (° C.), a reaction time of 20 minutes (min), and a mixing ratio of 50:100 of said dry weight of said rice straw to said weight of said solution.

6. The method according to claim 4, wherein, in step (b2), said mixing ratio is reduced to 30:100.

7. The method according to claim 4, wherein, in step (b2), said temperature is 130° C.

8. The method according to claim 4, wherein, in step (c2), said period of time is 15 min.

9. The method according to claim 1, wherein, in step (b1), said a final xylose concentration is 30 grams per liter (g/L) and said glucose concentration is 10 g/L.

10. The method according to claim 1, wherein, in step (c1), said volumetric ratio of said hydrolysate to said synthetic medium is gradually increased from said initially volumetric ratio of 20:80 to said final volumetric ratio of 70:30 along through 35:65, 50:50 and 60:40.

11. The method according to claim 1, wherein, in step (d1), said plurality of generations is 60 generations.

12. The method according to claim 1, wherein said cultivated yeast use for converting xylose into ethanol in the hydrolysate.

13. The method according to claim 12, Wherein said hydrolysate is obtained with a material selected from a group consisting of rice straw, bagasse, silvergrass, napiergrass, switchgrass, corn stover, wheat straw, wood, bamboo, water hyacinth and algae.

14. The method according to claim 12, wherein said hydrolysate is not detoxified and directly added with an alkaline agent to obtain a weak acidity solution.

15. The method according to claim 14, wherein said weak acidity has a pH value between 5.0 and 7.0.

16. The method according to claim 12, wherein said hydrolysate is processed through an overliming operation to be detoxified and become a weak acid solution.

17. The method according to claim 16, wherein said overliming operation comprises steps of:
(a3) heating up said hydrolysate to a temperature;
(b3) adding said hydrated lime to lead hydrolysate to be detoxified with gypsum obtained while a pH value of said hydrolysate is increased;
(c3) removing said gypsum from said hydrolysate with solid and liquid separation equipments; and
(d3) adding an acidic agent to obtain a weak acidity of said hydrolysate.

18. The method according to claim 17, wherein, in step (a3), said temperature is located between 50° C. and 60° C.

19. The method according to claim 17, wherein, in step (b3), said pH value of said hydrolysate is increased to a value between 9.0 and 11.0.

20. The method according to claim 17, wherein said weak acidity was a pH value between 5.0 and 7.0.
